(19) United States
(12) Patent Application Publication
(10) Pub. No.: US 2018/0201703 A1
(43) Pub. Date: Jul. 19, 2018

(54) VANILLIN METHACRYLATES AND POLYMERS THEREFROM

(71) Applicants: Washington State University, Pullman, WA (US); Iowa State University Research Foundation, Inc., Ames, WA (US)

(72) Inventors: Michael Richard Kessler, Fargo, ND (US); Chaoqun Zhang, Guangzhou (CN)

(73) Assignee: Iowa State University Research Foundation, Inc., Ames, IA (US)

(21) Appl. No.: 15/742,499

(22) PCT Filed: Jul. 7, 2016
(86) PCT No.: PCT/US16/41255

§ 371 Date: Jan. 5, 2018

Related U.S. Application Data
(60) Provisional application No. 62/189,625, filed on Jul. 7, 2015.

(51) Int. Cl. C08F 136/20; C07C 69/602

(52) U.S. Cl. CPC C08F 136/20; C07C 69/602

(57) ABSTRACT

Vanillin and vanillyl alcohol were modified into methacrylated derivatives. The structures of vanillin-based monomers were characterized by NMR and FTIR. Renewable polymers were prepared from these vanillin-based monomers. The effects of structure and functionality of the vanillin-based monomers on the thermo-mechanical properties of the resulting polymers were investigated and discussed. Polymers from methacrylated vanillyl alcohol (MVA) demonstrated greater storage moduli, higher glass transition temperatures, and thermal resistance than those from methacrylated vanillin (MV) because of the different functionalities of their monomers.

### CROSS-REFERENCE TO RELATED APPLICATION

This application claims the benefit under 35 U.S.C. § 119(e) of U.S. Provisional Patent Application No. 62/189,625 filed Jul. 7, 2015; where this provisional application is incorporated herein by reference in its entirety.

### FIELD OF THE INVENTION

The present invention relates generally to organic polymers, and more specifically to methacrylated vanillyl alcohol as a monomer or co-monomer for polymer preparation, and as a reactive diluent for polymer preparation, e.g., thermoset polymers.

### BACKGROUND

Unsaturated polyester resins (UPR) and vinyl ester resins (VER) are widely used thermosetting polymers for fiber reinforced composites. For example, global UPR market is approximately a 5,000 kilo ton business and is experiencing continued growth. Vinyl ester resins (VER) have been widely used as matrix materials for advanced polymer composites in various applications because of their excellent corrosion and degradation resistance, high glass transition temperature, high strength-to-weight ratios, and low cost (see, e.g., J. J. La Scala, et al., Polymer 2005, 46, 2908; E. T. Thostenson, et al., Compos Sci Technol 2009, 69, 801; and J. Zhu, et al., Compos Sci Technol 2007, 67, 1509).

Until recently, petrochemicals were the resource of choice for production of commodity monomers for vinyl ester resins. However, the continued utilization of these nonrenewable resources raises concerns regarding environmental pollution and depletion of nonrenewable resources (see, e.g., J. Zakzeski, et al., Chemsuschem 2012, 5, 1602; and C. Q. Zhang, et al., Acs Sustain Chem Eng 2014, 2, 2465). Also, widely used petroleum-based monomers, such as styrene, are often used as reactive diluents with both vinyl ester resins and unsaturated polyesters. However, such reactive diluents are often considered hazardous air pollutants (HAPs) and volatile organic compounds (VOCs) (see, e.g., J. J. La Scala, et al., Polymer 2004, 45, 7729; and J. F. Stanzione, et al., Acs Sustain Chem Eng 2013, 1, 419).

Unsaturated polyester resins (UPR) and vinyl ester resins (VER) typically are mixed with styrene (in amounts up to 50%) as a reactive diluent before being cured by a free radical polymerization. However, styrene offers significant disadvantages owing to health, safety, and environmental concerns. The Clean Air Act Amendments of 1990 lists styrene as a hazardous air pollutant and occupational exposure to styrene is regulated by the Occupational Safety and Health Administration (OSHA). Styrene is also derived from petroleum, a non-renewable resource.

There exists a need for alternatives to styrene which overcome one or more of the shortcoming associated with the prior art.

In order to develop sustainable and environmentally friendly vinyl ester resins, the identification of renewable building blocks that substitute petroleum-based components in these resins has seen increasing efforts. Several renewable resources (cellulose, starch, natural oils, etc.) have been exploited to produce novel bio-monomers for the development of polymeric materials (see, e.g., R. P. Wool and X. S. Sun, “Bio-based polymers and composites”, Elsevier Academic, Amsterdam; Oxford, 2005; A. Gandini, Macromolecules 2008, 41, 9491; and C. Q. Zhang, et al., Macromol Rapid Comm 2014, 35, 1068). However, most of these bio-monomers are aliphatic or cycloaliphatic, resulting in polymers with low structural rigidity and thermal stability (see, e.g., M. Fache, et al., Green Chem 2014, 16, 1987).

Recently, attention has turned to bio-based phenolic compounds, such as lignin model compounds (see, e.g., J. F. Stanzione, et al., Chemsuschem 2012, 5, 1291) and cashew nut shell liquid-derived aromatics (see, e.g., R. L. Quirino, et al., Green Chem 2014, 16, 1700), for high performance vinyl ester resins that exhibit similar or better properties than commercial petroleum-based products.

Vanillin, originally an extraction product of vanilla plantifolia beans, is one of the most widely used flavors in foods, fragrances, beverages, and pharmaceuticals (see, e.g., C. Brazinha, et al., Green Chem 2011, 13, 2197). Certain vanillin derivatives have been used as renewable building blocks for high performance polymers mainly because of their rigid aromatic structures (see, e.g., J. F. Stanzione, et al., Green Chem 2012, 14, 2346). The use of vanillin as a bio-resource for the production of novel polymeric materials is possible because it can be mass-produced from lignin, which is one of the most abundant feedstocks in nature, as wood contains approximately 30% lignin (see, e.g., D. M. Fries, et al., Chem Eng Technol 2008, 31, 1182; J. H. Lora and W. G. Glasser, J Polym Environ 2002, 10, 39; T. Voitl, P. R. von Rohr, Chemsuschem 2008, 1, 763; and L. Mialon, et al., Green Chem 2010, 12, 1704).

Vanillin has hydroxyl and aldehyde reactive sites that can be used for chemical modifications to produce monomers that can be polymerized into materials with different mechanical and thermal properties. For example, bisphenols prepared by hydrogenation of vanillin to creosol followed by condensation with various aldehydes can be converted to a series of renewable bis(cyanate) esters with high glass transition temperatures (219-248° C.) and good thermal stability up to 400° C. (see, H. A. Meylemans, et al., Biomacromolecules 2013, 14, 771) Electrochemical reductive polymerization of divanillin in aqueous sodium hydroxide resulted in polyvanillin (91% yield) with good thermal stability (see, A. S. Amarasekara, et al., Green Chem 2012, 14, 2395). Polymerization of acetyldihydroferulic acid, prepared by a Perkin reaction between vanillin and acetic anhydride followed by hydrogenation, leads to biorenewable poly(dihydroferulic acid), which exhibits properties similar to polyethylene terephthalate from commercial petroleum-based resources (see, L. Mialon, et al. Green Chem 2010, 12, 1704). Also, vanillin has been modified into methacrylated derivatives for vinyl ester resins. Renbutsu et al. successfully prepared methacrylated vanillin (MV) via Steglich esterification of vanillin with methacrylic acid as coating materials (see, E. Renbutsu, et al., Carbohyd Polym 2007, 69, 697). Patel et al. synthesized methacrylated vanillin by esterification between vanillin and methacryloyl chloride in order to produce polymers with potential antimicrobial applications (see, R. J. Patel, et al., Der Pharma Chemica 2013, 5, 63). Methacrylated vanillin was also prepared by the reaction of vanillin and methacrylic anhydride by Stanzione et al, Chemsuschem 2012, 5, 1291) and the vinyl ester resin resulting after copolymerization of methacrylated vanillin and glycerol dimethacrylate showed a high glass transition temperature (155° C.) and high storage modulus (see, J. F. Stanzione, et al., Green Chem 2012, 14, 2346). However, the functional aldehyde groups in all MV monomers, which are normally used to control polymer properties such as mechanical and thermal stability, are not exploited.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, in one aspect the invention relates to a monomer called methacrylated vanillyl alcohol (MVA), as a bio-based, environmentally-friendly alternative to styrene. The monomer has a low viscosity at room temperature, which facilitates its use as a reactive diluent to lower the viscosity of UPR and VER systems. In addition, MVA polymerizes into a highly crosslinked thermosetting polymer useful for the manufacture of various objects where an economical rigid plastic is desired, e.g., disposable plastic dinnerware and cutlery, smoke detector housings, and plastic model assembly kits, to name a few. For comparison, MVA homopolymerizes into a plastic with a T_{g} of about 130° C., while polystyrene has a T_{g} of 100° C.

MVA is straight-forward to prepare. The starting material, vanillyl alcohol, may be prepared by the reduction of vanillin, and is a commercially available material. Vanillyl alcohol may be reacted with methacrylic anhydride in the presence of a suitable catalyst such as 4-methylaminopyridine as catalyst to make the methacrylated vanillylalcohol (MVA). It has been discovered that the resulting MVA is a low-viscosity liquid at room temperature, which is in contrast to the properties of methacrylated vanillin (MV), which is solid at room temperature and thus MV is not suitable as a reactive diluent. It has also been found that polymers from MVA demonstrate greater storage moduli, higher glass transition temperatures, and greater thermal resistance than polymers prepared from MV. Thus, unlike MV, this new monomer has utility as a bio-based reactive diluent for unsaturated polyester resins and vinyl esters to replace styrene.

Vanillin is a readily available material. For example, vanillin is a naturally occurring chemical which may be extracted from vanilla beans. According to Frache et al, Borregaard, the second largest vanillin producer in the world, has a commercial process to isolate vanillin from lignin (see, M. Fache, et al., Green Chem 2014, 16, 1987). Vanillin is widely used in flavoring food. The present invention recognizes that vanillin is a very attractive feedstock for bio-based chemicals and polymers.

In exemplary embodiments, the present disclosure provides:

A compound named MVA of formula

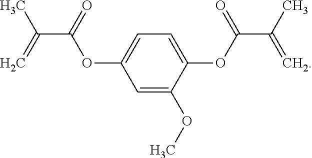 [SMILES: C=C(C)C(=O)Oc1ccc(OC(=O)C(=C)C)c(OC)c1]

A homopolymer formed by free radical initiated polymerization of a compound of formula

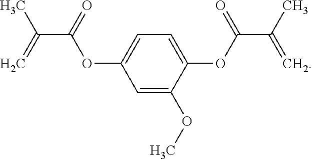 [SMILES: C=C(C)C(=O)Oc1ccc(OC(=O)C(=C)C)c(OC)c1]

A copolymer formed by free radical initiated polymerization of a compound of formula

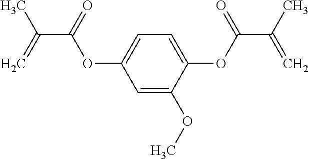 [SMILES: C=C(C)C(=O)Oc1ccc(OC(=O)C(=C)C)c(OC)c1]

and a co-monomer that also undergoes free radical initiated polymerization.

A polymer comprising a plurality of structural units of formulae

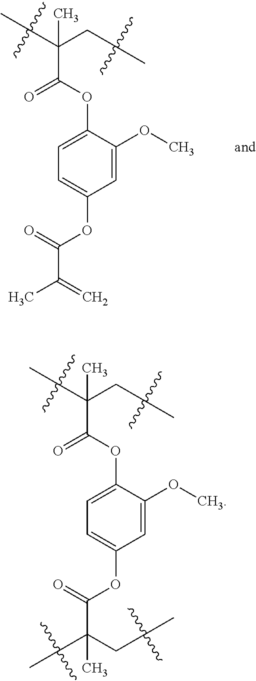

A thermoset unsaturated polyester resin prepared from reactants comprising an unsaturated compound of formula

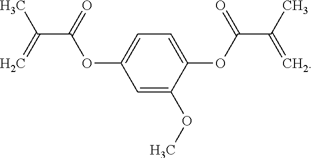 [SMILES: C=C(C)C(=O)Oc1ccc(OC(=O)C(=C)C)c(OC)c1]

A thermoset vinyl ester resin prepared from reactants comprising an unsaturated compound of formula

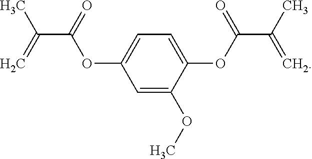 [SMILES: C=C(C)C(=O)Oc1ccc(OC(=O)C(=C)C)c(OC)c1]

In a method for preparing a polymer from monomers comprising styrene, the improvement comprising replacing at least some of the styrene with a compound of formula

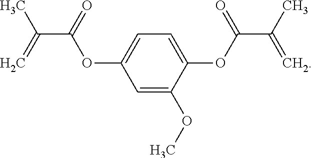 [SMILES: C=C(C)C(=O)Oc1ccc(OC(=O)C(=C)C)c(OC)c1]

A homopolymer formed by free radical initiated polymerization of a compound of formula

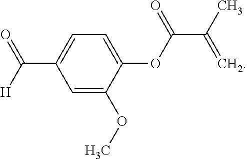 [SMILES: C=C(C)C(=O)Oc1ccc(C=O)cc1OC]

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying Figures and the following detailed description of various embodiments. Non-limiting and non-exhaustive embodiments are described with reference to the accompanying Figures. One or more embodiments are described hereinafter with reference to the accompanying Figures in which:

FIG. 1 shows 1H NMR spectra of vanillin and selected derivatives thereof.

FIG. 2 shows FTIR spectra of vanillin and selected derivatives thereof.

FIG. 3(a) shows the time dependence of G′ and G″ for cure processes of MV.

FIG. 3(b) shows the time dependence of G′ and G″ for cure processes of MVA.

FIG. 4(a) shows storage moduli of two vanillin-based polymers as functions of temperature.

FIG. 4(b) shows loss moduli of two vanillin-based polymers as functions of temperature.

FIG. 5 shows TGA curves and their derivative curves of polymers from MV and MVA in nitrogen.

FIG. 6 shows schemes for the preparation of MV and MVA from vanillin.

### DETAILED DESCRIPTION OF THE INVENTION

The present invention may be understood more readily by reference to the following detailed description of preferred embodiments of the invention and the Examples included herein.

In one aspect, the present invention is directed to renewable polymers prepared from vanillin and its derivatives. Vanillin and vanillyl alcohol may be modified into methacrylated derivatives, which are subsequently polymerized by a free-radical process. The rheokinetics of the polymerization are described herein in order to understand the cure behavior and optimize the polymerization conditions for these two monomers. The effect of both structure and functionalities of the vanillin-based monomers on the thermo-mechanical properties of the resulting polymers are also provided. The high cross-linking density of the polymers from methacrylated vanillyl alcohol results in higher storage modulus and glass transition temperature, as well as better thermal resistance, than seen in polymers from methacrylated vanillin. These properties, combined with methacrylated vanillyl alcohol's low-viscosity at room temperature, make it useful as a bio-based reactive diluent for unsaturated polyester resins and vinyl esters.

According to the present disclosure, methacrylated vanillin (MV) and methacrylated vanillyl alcohol (MVA) are prepared and polymerized via free-radical polymerization to produce novel renewable polymers. The structure of MV and MVA were characterized by proton nuclear magnetic resonance (¹H NMR) and Fourier transform infrared spectroscopy (FTIR). In order to describe the cure behavior and optimize the polymerization conditions for these two vanillin-based monomers, a dynamic rheology study was carried out by small-amplitude oscillatory shear flow experiments and used to monitor physical and chemical crosslinking reactions and microstructure changes during cure processing. The resulting polymers were characterized by dynamic mechanical analysis (DMA), differential scanning calorimetry (DSC), and thermogravimetric analysis (TGA). The effects of chemical structure and composition of the monomers on the thermomechanical properties of the resulting polymers are described.

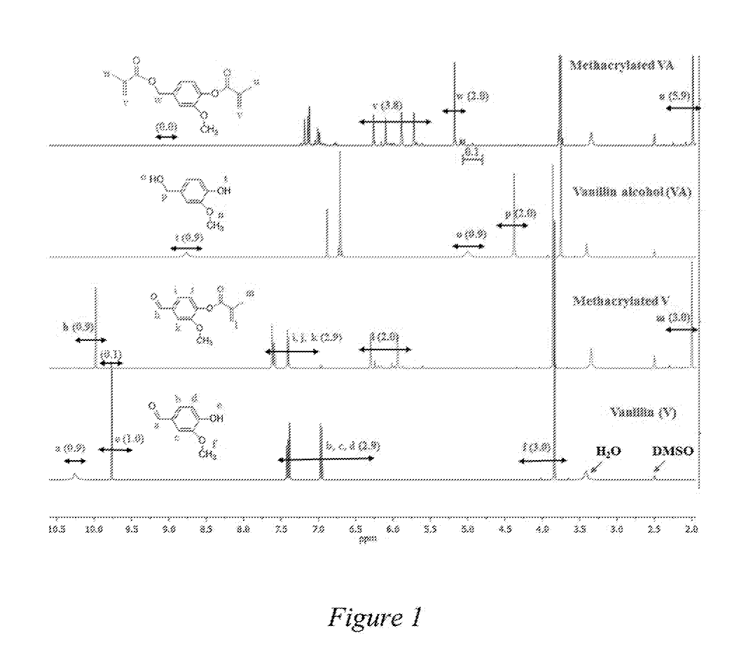

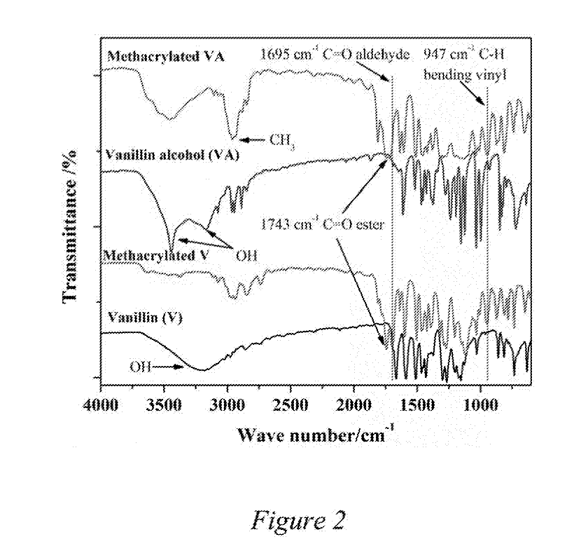

The ¹H NMR spectra of vanillin and its derivatives are shown in FIG. 1. Using the area under the peaks at 3.5-4.0 ppm (the terminal O—CH₃ attached to the benzene ring) of all vanillin and its derivatives for normalization (with an integrated value of 3), the numbers of protons associated with the functional groups are shown in parentheses. Compared to the spectrum of vanillin, the disappearance of the peaks at 9.7-9.8 ppm in MV indicated the consumption of hydroxyl groups attached to aromatic rings. The new peaks at 5.7-6.5 indicated the vinyl groups in MV. These results confirmed the reaction between vanillin and methacrylate anhydride and that the conversion of hydroxyl groups into vinyl groups was almost 100%. Similar observations were made for MVA from vanillyl alcohol. The disappearance of the peaks at 8.5-8.7 ppm and 4.7-5.1 ppm, and the appearance of peaks at 5.5-6.2 confirmed the successful preparation of MVA. The functionality of MV (peaks at 5.7-6.5) is 1 and that of MVA is close to 2 (peaks at 5.5-6.2). The disappearance of the 3300 cm⁻¹ peak, and the appearance of the 1743 cm⁻¹ and 947 cm⁻¹ peaks in the FT-IRs of MV and MVA also confirmed the above conclusions (see FIG. 2).

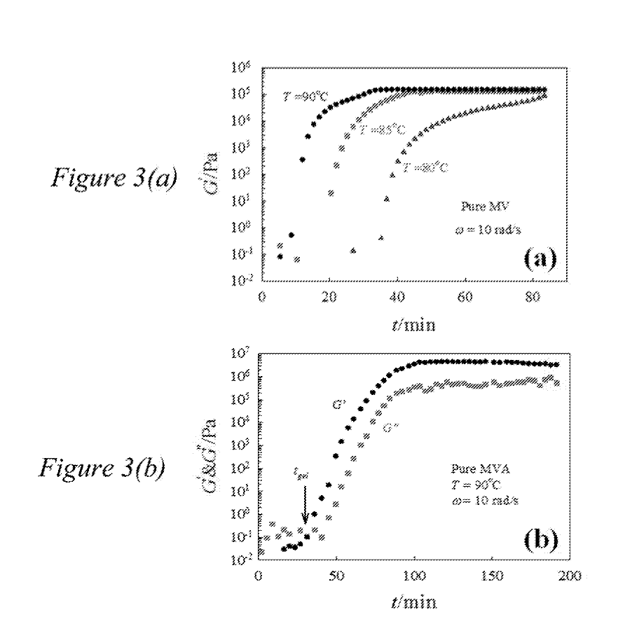

FIG. 3(a) shows the isothermal time dependence of G′ at different constant temperatures and a constant angular frequency of ω=10 rad/s for MV. A dramatic increase in G′ was observed during the early stages of the curing process, followed by a plateau of the G′ values at longer cure times. The magnitudes of the increase in G′ before reaching the plateau was strongly temperature-dependent. Increasing temperatures, even by as little as from 80 to 90° C., greatly increased the increase in G′, which can be attributed to significant chain growth increases caused by higher temperatures, resulting in shorter cure times before arriving at the plateau region. The higher temperatures increase the formation of free radicals, initiating simultaneous growth of more polymer chains and therefore resulting in shorter chains. Also the initial increase in G′ noticed at higher temperatures is related to a faster reaction rate.

FIG. 3(b) shows the time dependence of G′ and G″ at 90° C. and ω=10 rad/s for MVA. At the beginning of the cure process, G′ was one order of magnitude lower than G″, demonstrating the liquid-like behavior of the samples. Both G′ and G″ increased with time, but G′ increased more rapidly than G″. At the late stages of the cure process, G′ had increased to values one order of magnitude greater than G″, indicating the formation of cross-linked structures. The value of T_{gel}=40 min was calculated from the crossover point of G′ and G″, as indicated by the arrow in FIG. 3(b). The G′ values of MVA reached the plateau region after approx. 110 min at 90° C. The G′ values for MVA at the plateau region are higher than the values for MV because of the higher crosslinking density of polymers from MVA.

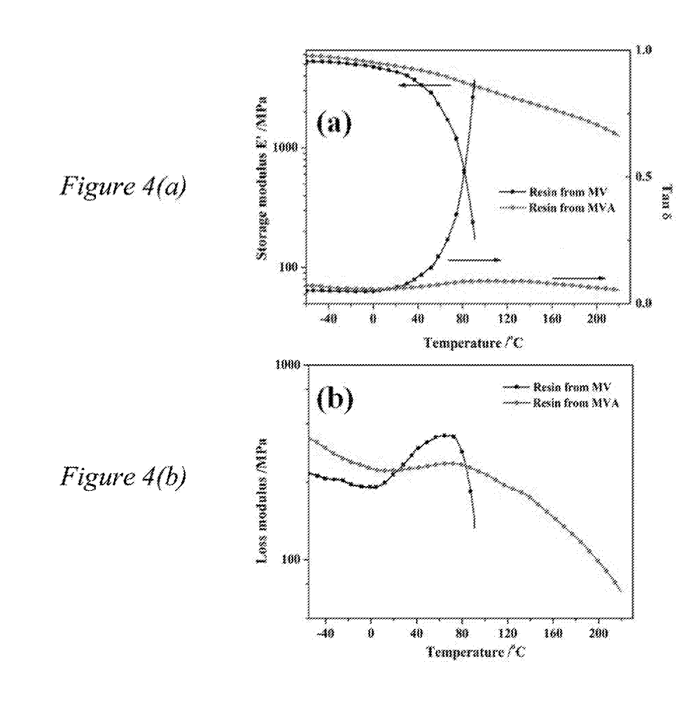

The thermo-mechanical properties of the vanillin-based resins as described herein were measured using DMA; the storage and loss moduli and tan δ are shown as functions of temperature in FIG. 4. Generally, the viscoelastic properties of a polymer are characterized by its storage modulus as the elastic portion, indicating the stored energy during a cyclic load, while the loss modulus represents the viscous portion, indicating the energy dissipated through cyclic loading. The ratio of these two moduli (loss modulus/storage modulus) is tan δ, also called the loss tangent. FIG. 4 shows that the storage moduli of both films were strongly temperature-dependent; i.e., they demonstrated a glassy state below room temperature. A slight decrease in storage moduli was observed with increasing temperature up to 40° C., while a sharp decrease was seen above 40° C. Resins based on MVA and MV were both hard and rigid polymers, similar to commercial vinyl ester polymers (see, e.g., J. J. La Scala, et al., Polymer 2004, 45, 7729).

The resins from MVA showed a higher storage modulus and a lower rate of decrease in storage modulus with increasing temperatures. The storage modulus values for both films were similar at 25° C., while at 80° C. the resin from MVA showed a much higher storage modulus than that from MV. The resin from MVA showed a peak in tan δ at 131.6° C. and a corresponding peak in loss modulus at 83.3° C., while the resin from MV demonstrated a peak in the loss modulus at 68.9° C. The peak in tan δ for resins from MV was fully undetectable because of their brittle nature. However, FIG. 4 shows that the height of the tan δ peaks for resins from MVA was much lower than that for resin from MV, indicating the higher cross-linking densities for MVA derived film (see, e.g., C. Q. Zhang, et al., Green Chem 2013, 15, 1477).

Because with increasing cross-linking densities, molecular motions of polymer chains become more restricted and the amount of energy that can be dissipated throughout the polymers decreases dramatically, therefore, the tan δ peak shifts to a higher temperature and the (tan δ)max decreases. Both the temperatures at which the peak of the loss modulus and the peak of tan δ occurred are indicative of the glass transition temperature. The glass transition temperature of both films measured by DSC showed the similar trend as shown Table 1.

TABLE 1

| | | | DMA T_{g} | | | TGA in | |
|---|---|---|---|---|---|---|---|
| | Storage moduli | | (° C.) | | DSC | nitrogen | |
| | (GPa) | | | Loss | T_{g} | (° C.) | |
| | 25° C. | 80° C. | Tan δ | moduli | (° C.) | T₁₀ | T₅₀ |
| Resin from MV | 4.2 | 0.7 | 90.5 | 68.9 | 72 | 280 | 418 |
| Resin from | 4.7 | 3.5 | 131.6 | 83.3 | 99.1 | 341 | 438 |
| MVA | | | | | | | |

The inventors speculate that two possible reasons contributed to the facts that resins from MVA demonstrated higher storage moduli and higher T_{g} than those made from MV. First, MV is mono-functional, while MVA is di-functional. Thus, resin from MVA exhibited higher crosslinking densities than those from MV, resulting in more restrictions to molecular motion of the polymer chains. Second, in the resin from MVA, all aromatic molecules are incorporated into the polymer networks, resulting in enhanced structural rigidity, while the rigid aromatic rings in the resins from MV act as dangling chains along the polymer molecule.

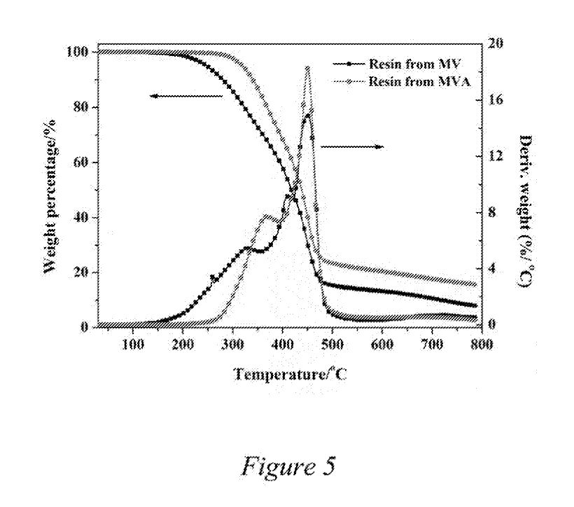

FIG. 5 shows the TGA curves and their derivatives for resins from MV and MVA in nitrogen. The derivative curves indicate that the degradation of the polymers occurs in two stages: in a lower temperature range (200-390° C.) and a higher temperature range (390-500° C.). The former stage corresponds to the decomposition initiated at the unsaturated chain ends of the polymers and the degradation of potential oligomers (see, J. F. Stanzione, et al., Green Chem 2012, 14, 2346). The latter is attributed to the degradation initiated by random scission of polymers at high temperatures (see, e.g., S. Zulfigar, et al., Polym Degrad Stabil 1997, 55, 257). Because of the higher unsaturation of MVA, its resulting polymers exhibit higher decomposition resistance in the lower temperature range. The high crosslinking density of MVA polymers restricts their depolymerization by rearrangement, leading to high decomposition resistance in the higher temperature range. Also, the incorporation of stable aromatic rings into the network of MVA thermosets increased the thermal stability of the final resins in the second stage compared to MV thermosets. Table 1 summarizes the 10 and 50% degradation for the two different thermosets.

The present disclosure provides two methacrylated derivatives from vanillin and vanillyl alcohol, and a solvent-free method for preparing vinyl ester resins. The rheokinetics of the polymerization were investigated to determine the cure behavior and optimize the free-radical polymerization conditions for these two monomers. The thermo-mechanical behaviors of these renewable resins indicated that they are useful for polymer composite applications.

For example, in one embodiment the present disclosure provides a compound of formula

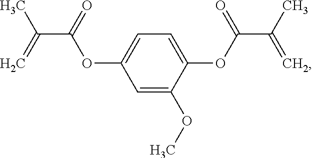 [SMILES: C=C(C)C(=O)Oc1ccc(OC(=O)C(=C)C)c(OC)c1]

which is referred to herein as methacrylated vanillyl alcohol (MVA). In addition, the present disclosure provides a homopolymer formed by free radical initiated polymerization of MVA, where that polymerization may be a bulk polymerization process, i.e., conducted in the absence of a solvent. MVA may be used in polymerization reactions that include co-monomer(s) that also undergo free radical initiated polymerization (e.g., styrene, other acrylates) to form a copolymer. The polymers prepared from MVA, as referred to herein as resins, may comprise a plurality of structural units derived from MVA, e.g., structural units of formulae

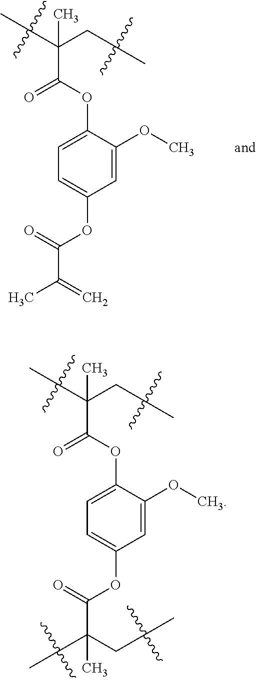

Thus, the present disclosure provides polymers/resins that are prepared in whole or in part from MVA. For example, a homopolymer formed by free radical initiated polymerization of MVA. As another example, a thermoset unsaturated polyester resin prepared from reactants that include MVA. In yet another example, a thermoset vinyl ester resin prepared from reactants that include MVA. In one embodiment, some or all of the styrene that is used in a process for resin manufacture may be replaced with MVA. Thus, the present disclosure provides a method for preparing a polymer from monomers comprising styrene, the improvement comprising replacing at least some of the styrene with MVA.

The present disclosure also provides a homopolymer formed by free radical initiated polymerization of a compound of formula

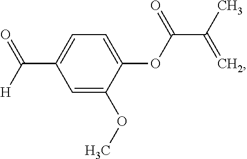 [SMILES: C=C(C)C(=O)Oc1ccc(C=O)cc1OC]

which is referred to herein a MV. The free radical initiated polymerization may be a bulk polymerization process, i.e., a polymerization process that does not include a solvent.

### Experimental Section

Vanillin (assay: 99%), vanillyl alcohol (assay: >98%), methacrylic anhydride, magnesium sulfate (MgSO₄), sodium bicarbonate, methylene chloride, 4-dimethylaminopyridine (DMAP), and N-tert-butyl peroxybenzoate (TBPB) were purchased from Sigma-Aldrich (Milwaukee, Wis.). All materials were used as received without further purification.

Rheokinetics studies of the cure process were carried out by small-amplitude oscillatory shear flow experiments for two systems: (1) MV with 2 wt. % TBPB catalyst, (2) MVA with 2 wt. % TBPB catalyst. All isothermal measurements were conducted using TA instruments (AR2000ex) to determine the influence of the cure process on the viscoelastic characteristics (G′ and G″). Standard procedures were followed: a time sweep at different constant temperatures (80, 85, 90° C.) and constant angular shear frequency (ω=10 rad/s).

The chemical structures of vanillin and its derivatives were analyzed by ¹H NMR spectroscopy using a Varian spectrometer (Palo Alto, Calif.) at 300 MHz and by FT-IR spectroscopy using a Nicolet 460 FT-IR spectrometer (Madison, Wis.).

The thermo-mechanical properties of the resins were evaluated using a TA Instruments Q800 DMA in three point bending mode at 1 Hz. Rectangular specimens (1.2 mm thickness×8 mm width) were used. The samples were cooled and equilibrated for 3 min at −50° C., then heated to 210° C. at a rate of 3° C./min. A TA Instruments Q2000 DSC was used to determine the glass transition temperatures (T_{g}). Samples of approximately 7 mg were heated from room temperature to 170° C. at a rate of 20° C./min to erase their thermal history. Then the samples were equilibrated at −60° C., followed by a second heating cycle to 170° C. at a heating rate of 20° C./min. The thermal stability of the resins was evaluated using a TA Instruments Q50 TGA. Samples with weights of approx. 10 mg were heated from room temperature to 800° C. at a heating rate of 20° C./min under a nitrogen atmosphere. These renewable resins prepared using a solvent-free method are suitable for use in polymer composite applications.

### Example 1: Preparation of Methacrylated Vanillin (MV)

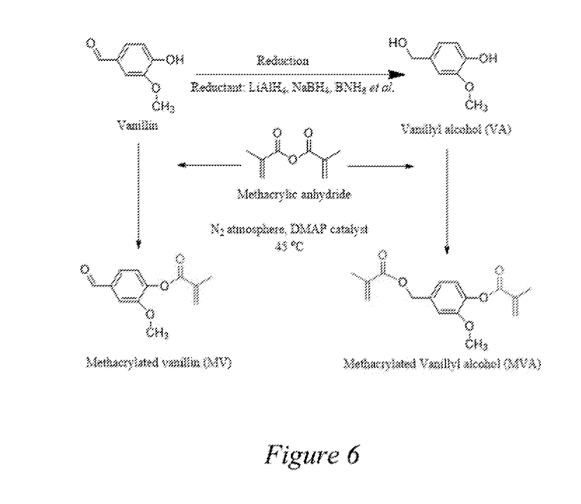

Vanillin was charged into a 500 ml flask. Methacrylic anhydride and DMAP were added into the mixture under vigorous stirring. The mole ratio of the hydroxyl group and the anhydride group was 1:1.1. The mixture was allowed to react at 50° C. for 18 h under nitrogen atmosphere. Then, sodium bicarbonate was added to the mixture to neutralize the reactants until gas evolution ended. Methylene chloride was added to extract the organic layer. The organic layer was washed with sodium bicarbonate solution four times. Methacrylated vanillin was obtained after drying with MgSO4, filtering, removal of organic solvent by rotary evaporation, and drying in a vacuum oven at 80° C. overnight. See, e.g., J. F. Stanzione, et al., Chemsuschem 2012, 5, 1291, and FIG. 6.

Thus, in one aspect the present disclosure provides a method for preparing methyacrylated vanillin by reaction of vanillin and methacrylic anhydride in the presence of a suitable base such as DMAP.

### Example 2: Preparation of Methacrylated Vanillyl Alcohol (MVA)

Vanillyl alcohol can be prepared by the reduction of aldehyde groups in vanillin. Several catalytic hydrogenation agents, including LiAIH₄, ammonia borane, and sodium borohydride, can be used as reducing agents (see, e.g., A. R. Baru and R. S. Mohan, J Chem Educ 2005, 82, 1674). Alternatively, commercially available vanillyl alcohol may be used. Methacrylated vanillyl alcohol (MVA) was prepared in analogy to the preparation of methacrylated vanillin (MV) as described in Example 1. Noteworthy is that MV is solid, while MVA is a low-viscosity liquid at room temperature. Thus, MVA is a viable reactive diluent for renewable vinyl ester resins in polymer composite applications while MV is not suitable, as previously reported (see, J. F. Stanzione, et al., Chemsuschem 2012, 5, 1291 and FIG. 6).

Thus, in one aspect the present disclosure provides a method for preparing methyacrylated vanillyl alcohol by reaction of vanillyl alcohol and methacrylic anhydride in the presence of a suitable base such as DMAP.

### Example 3: Synthesis of Vanillin-Based Resins from MVA

Renewable vanillin-based polymers were prepared in silicone molds by bulk polymerization of MVA at 90° C. for 2 h and 130° C. for 2 h, respectively. TBPB (2 wt. %) was used as the free radical initiator. The polymerization process was carried out under a nitrogen atmosphere. The resulting resins were cut into specific dimensions for thermo-mechanical testing, with results as discussed herein.

Thus, in one aspect the present disclosure provides a method for preparing a resin comprising combining MVA and a suitable free radical initiator, optionally in the absence of a solvent, i.e., by bulk polymerization.

### Example 4: Synthesis of Vanillin-Based Resins from MV

Renewable vanillin-based polymers were prepared in silicone molds by bulk polymerization of MV 90° C. for 2 h and 130° C. for 2 h, respectively. TBPB (2 wt. %) was used as the free radical initiator. The polymerization process was carried out under a nitrogen atmosphere. The resulting resins were cut into specific dimensions for thermo-mechanical testing, with results as discussed herein.

Thus, in one aspect the present disclosure provides a method for preparing a resin comprising combining MV and a suitable free radical initiator, optionally in the absence of a solvent, i.e., by bulk polymerization.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

In the foregoing description, certain specific details are set forth to provide a thorough understanding of various disclosed embodiments. However, one skilled in the relevant art will recognize that embodiments may be practiced without one or more of these specific details, or with other methods, components, materials, etc. The Examples and preparations provided herein further illustrate and exemplify the compounds and polymer of the present invention and methods of preparing such compounds and polymers. It is to be understood that the scope of the present invention is not limited in any way by the scope of the Examples and preparations.

Although any methods and materials similar or equivalent to those described herein can also be used in the practice or testing of the present invention, a limited number of the exemplary methods and materials are described herein.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated.

The inventors have provided various speculation herein concerning reaction conditions and other matters pertaining to the reactivity of MV and MVA. The inventors are not bound to that speculation.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A compound of formula

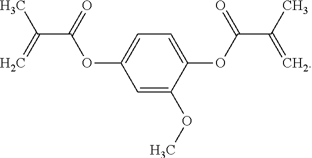 [SMILES: C=C(C)C(=O)Oc1ccc(OC(=O)C(=C)C)c(OC)c1]

2. A homopolymer formed by free radical initiated polymerization of the compound of claim 1.

3. A copolymer formed by free radical initiated polymerization of the compound of claim 1 and a co-monomer.

4. A thermoset unsaturated polyester resin prepared from reactants comprising the compound of claim 1.

5. A thermoset vinyl ester resin prepared from reactants comprising the compound of claim 1.

6. In a method for preparing a polymer from monomers comprising styrene, the improvement comprising replacing at least some of the styrene with a compound of claim 1.

7. A homopolymer formed by free radical initiated polymerization of a compound of formula

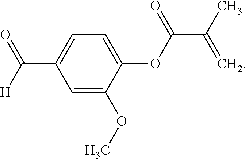 [SMILES: C=C(C)C(=O)Oc1ccc(C=O)cc1OC]

8. A polymer comprising a plurality of structural units of formulae [chemical structure]
